(19) United States
(12) Patent Application Publication
(10) Pub. No.: US 2011/0160687 A1
(43) Pub. Date: Jun. 30, 2011

(54) NONWOVEN COMPOSITE INCLUDING AN APERTURED ELASTIC FILM AND METHOD OF MAKING

(76) Inventors: Howard M. Welch, Woodstock, GA (US); Glenda J. Rhodes, Stone Mountain, GA (US); Lei Huang, Duluth, GA (US); Matthew B. Lake, Cumming, GA (US); Wing-Chak Ng, Suwanee, GA (US); Akwokwo H. Redhead, Atlanta, GA (US); Jose Augusto Vidal de Siqueira, Roswell, GA (US); Oomman P. Thomas, Alpharetta, GA (US)

(21) Appl. No.: 12/649,508

(22) Filed: Dec. 30, 2009

(51) Int. Cl. A61F 13/53; B32B 38/00; B32B 38/04; B32B 3/24

(52) U.S. Cl. 604/367; 156/229; 156/192; 428/137

(57) ABSTRACT

An elastic nonwoven composite that contains an elastic film laminated to one or more nonwoven web materials is provided. The composite is formed by passing an extrusion-coated film/nonwoven laminate through a nip to create apertures through both the film and the nonwoven. The apertures are of a size sufficient to provide a desired level of texture, softness, hand feel, and/or aesthetic appeal to the composite without having a significant adverse effect on its elastic properties. Apertures are accomplished in the present invention by selectively controlling certain parameters of the lamination process, such as film content, element pattern, degree of film tension, temperature and pressure conditions, and so forth.

### BACKGROUND OF THE INVENTION

Elastic composites are commonly incorporated into products (e.g., diapers, training pants, garments, etc.) to improve their ability to better fit the contours of the body. For example, the elastic composite may be formed from an elastic film and one or more nonwoven web materials. The elastic film may be coextruded onto the nonwoven web material to allow the use of particularly lightweight nonwoven materials. The resulting elastic composite is stretchable to the extent that the nonwoven web material is extensible. Unfortunately, elastic films often have unpleasant tactile aesthetic properties, such as feeling rubbery or tacky to the touch, making them unpleasant and uncomfortable against the wearer's skin. In an effort to improve the properties of elastic films, the films may be apertured. However, a need remains for improvement in methods of aperturing elastic film/nonwoven composites after formation of the composite material.

### SUMMARY OF THE INVENTION

In accordance with one embodiment of the present invention, a method of forming a nonwoven composite is disclosed. The method comprises forming an elastic film from a polymer composition on a nonwoven web material to form a film/nonwoven composite, stretching the film/nonwoven composite at a stretch ratio of about 1.5 or more, and passing the film/nonwoven composite through a nip formed by at least one patterned roll. At the nip, the film and the nonwoven web material are concurrently formed with apertures. In one aspect, the stretching may occur in either a machine direction or a cross-direction. Desirably, at least one of the apertures has a length of from about 200 to about 5000 micrometers. In one aspect, the apertures are defined by an aperture perimeter that defines a film flap extending at least partially across the aperture.

Other features and aspects of the present invention are described in more detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 schematically illustrates a method for forming a composite according to one embodiment of the present invention;

FIG. 2 illustrates one embodiment of an “S-weave” bonding pattern that may be used in accordance with the present invention;

FIG. 3 illustrates one embodiment of a “rib-knit” bonding pattern that may be used in accordance with the present invention;

FIG. 4 illustrates one embodiment of a “wire-weave” bonding pattern that may be used in accordance with the present invention;

FIG. 5 is a perspective view of grooved rolls that may be used in one embodiment of the present invention; and

FIG. 6 is a cross-sectional view showing the engagement between two of the grooved rolls of FIG. 5;

FIG. 7 is a perspective view of a personal care product that may be formed in accordance with one embodiment of the present invention;

FIG. 8 is an SEM microphotograph of an exemplary sample, showing apertures in an elastic laminate.

Repeat use of reference characters in the present specification and drawings is intended to represent same or analogous features or elements of the invention.

### DETAILED DESCRIPTION OF REPRESENTATIVE EMBODIMENTS

Reference now will be made in detail to various embodiments of the invention, one or more examples of which are set forth below. Each example is provided by way of explanation, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention cover such modifications and variations.

As used herein the term “nonwoven web” generally refers to a web having a structure of individual fibers or threads which are interlaid, but not in an identifiable manner as in a knitted fabric. Examples of suitable nonwoven fabrics or webs include, but are not limited to, meltblown webs, spunbond webs, bonded carded webs, airlaid webs, coform webs, hydraulically entangled webs, and so forth.

As used herein, the term “meltblown web” generally refers to a nonwoven web that is formed by a process in which a molten thermoplastic material is extruded through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “spunbond web” generally refers to a web containing small diameter substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is described and illustrated, for example, in U.S. Pat. Nos. 4,340,563 to Appel, et al., 3,692,618 to Dorschner, et al., 3,802,817 to Matsuki, et al., 3,338,992 to Kinney, 3,341,394 to Kinney, 3,502,763 to Hartman, 3,502,538 to Levy, 3,542,615 to Dobo, et al., and 5,382,400 to Pike, et al., which are incorporated herein in their entirety by reference thereto for all purposes. Spunbond fibers are generally not tacky when they are deposited onto a collecting surface. Spunbond fibers may sometimes have diameters less than about 40 microns, and are often between about 5 to about 20 microns.

As used herein, the terms “machine direction” or “MD” generally refers to the direction in which a material is produced. The term “cross-machine direction” or “CD” refers to the direction perpendicular to the machine direction.

As used herein the terms “extensible” or “extensibility” generally refers to a material that stretches or extends in the direction of an applied force by at least about 25%, in further embodiments about 50%, and in even further embodiments, at least about 75% of its relaxed length or width. An extensible material does not necessarily have recovery properties. For example, an elastomeric material is an extensible material having recovery properties. A meltblown web may be extensible, but not have recovery properties, and thus, be an extensible, non-elastic material.

As used herein, the term “elastomeric” and “elastic” and refers to a material that, upon application of a stretching force, is stretchable in at least one direction (such as the CD direction), and which upon release of the stretching force, contracts/returns to approximately its original dimension. For example, a stretched material may have a stretched length that is at least 50% greater than its relaxed unstretched length, and which will recover to within at least 50% of its stretched length upon release of the stretching force. A hypothetical example would be a one (1) inch sample of a material that is stretchable to at least 1.50 inches and which, upon release of the stretching force, will recover to a length of not more than 1.25 inches. Desirably, the material contracts or recovers at least 50%, and even more desirably, at least 80% of the stretched length.

As used herein, the terms “necked” and “necked material” generally refer to any material that has been drawn in at least one dimension (e.g., machine direction) to reduce its transverse dimension (e.g., cross-machine direction) so that when the drawing force is removed, the material may be pulled back to its original width. The necked material generally has a higher basis weight per unit area than the un-necked material. When the necked material is pulled back to its original width, it should have about the same basis weight as the un-necked material. This differs from the orientation of a film in which the film is thinned and the basis weight is reduced. The necking method typically involves unwinding a material from a supply roll and passing it through a brake nip roll assembly driven at a given linear speed. A take-up roll or nip, operating at a linear speed higher than the brake nip roll, draws the material and generates the tension needed to elongate and neck the material.

As used herein, the term “thermal point bonding” generally refers to a process performed, for example, by passing a material between a patterned roll (e.g., calender roll) and another roll (e.g., anvil roll), which may or may not be patterned. One or both of the rolls are typically heated.

As used herein, the term “ultrasonic bonding” generally refers to a process performed, for example, by passing a material between a sonic horn and a patterned roll (e.g., anvil roll). For instance, ultrasonic bonding through the use of a stationary horn and a rotating patterned anvil roll is described in U.S. Pat. Nos. 3,939,033 to Grgach, et al., 3,844,869 to Rust Jr., and 4,259,399 to Hill, which are incorporated herein in their entirety by reference thereto for all purposes. Moreover, ultrasonic bonding through the use of a rotary horn with a rotating patterned anvil roll is described in U.S. Pat. Nos. 5,096,532 to Neuwirth, et al., 5,110,403 to Ehlert, and 5,817,199 to Brennecke, et al., which are incorporated herein in their entirety by reference thereto for all purposes. Of course, any other ultrasonic bonding technique may also be used in the present invention.

Generally speaking, the present invention is directed to a nonwoven composite that contains an elastic film extrusion coated to one or more nonwoven web materials. The composite is formed by passing the extrusion-coated film/nonwoven laminate through a nip to create apertures in both the film and the nonwoven. The apertures are of a size sufficient to provide a desired level of texture, softness, hand feel, and/or aesthetic appeal to the composite without having a significant adverse effect on its elastic properties. Aperture formation is accomplished in the present invention by selectively controlling certain parameters of the lamination process, such as film content, pin pattern, degree of film tension and/or extension, temperature and pressure conditions, etc. In this regard, various embodiments of the present invention will now be described in more detail.

### I. Nonwoven Facing

As stated above, the nonwoven facing of the present invention is generally lightweight and has a low degree of strength in the cross-machine direction (“CD”), which increases the flexibility of the composite and also provides significant costs savings in its manufacture. More specifically, the basis weight may range from about 45 grams per square meter or less, in further embodiments from about 1 to about 30 grams per square meter, and in even further embodiments, from about 2 to about 20 grams per square meter. Likewise, the nonwoven facing may have a peak load in the cross-machine direction of about 350 grams-force per inch (width) or less, in further embodiments about 300 grams-force per inch or less, in even further embodiments from about 50 to about 300 grams-force per inch, in even further embodiments from about 60 to about 250 grams-force per inch, and in even further embodiments, from about 75 to about 200 grams-force per inch. If desired, the nonwoven facing may also have a low strength in the machine direction (“MD”), such as a peak load in the machine direction of about 3000 grams-force per inch (width) or less, in further embodiments about 2500 grams-force per inch or less, in even further embodiments from about 50 to about 2000 grams-force per inch, and in even further embodiments, from about 100 to about 1500 grams-force per inch.

The strip tensile strength values may be determined in substantial accordance with ASTM Standard D-5034. Specifically, a sample is cut or otherwise provided with size dimensions that measures 1 inch (25.4 millimeters) (width)×6 inches (152.4 millimeters) (length). A constant-rate-of-extension type of tensile tester is employed, such as, for example, a Sintech Tensile Tester, which is available from MTS Corp. of Eden Prairie, Minn. An appropriate load cell is selected so that the tested value falls within the range of 10-90% of the full scale load. The sample is held between grips having a front and back face measuring 1 inch (25.4 millimeters)×3 inches (76 millimeters). The grip faces are rubberized, and the longer dimension of the grip is perpendicular to the direction of pull. The grip pressure is maintained at a pressure of 60 to 80 pounds per square inch. The tensile test is run at a 20 inches per minute rate with a gauge length of 4 inches and a break sensitivity of 40%. Three samples are tested along the direction of interest. For example, the ultimate tensile strength (“peak load”), and peak elongation may be recorded.

The nonwoven facing may be formed from a variety of known processes, such as meltblowing, spunbonding, carding, wet laying, air laying, coform, etc. In one particular embodiment, for example, the nonwoven facing is a meltblown facing that contains “microfibers” in that they have an average size of about 15 micrometers or less, in further embodiments from about 0.01 to about 10 micrometers, and in even further embodiments, from about 0.1 to about 5 micrometers.

Regardless of the manner in which it is formed, the nonwoven facing is typically formed from a polymer having a relatively high Vicat softening temperature (ASTM D-1525), such as from about 100° C. to about 300° C., in further embodiments from about 120° C. to about 250° C., and in even further embodiments, from about 130° C. to about 200° C. Exemplary high-softening point polymers for use in forming nonwoven facings may include, for instance, polyolefins, e.g., polyethylene, polypropylene, polybutylene, etc.; polytetrafluoroethylene; polyesters, e.g., polyethylene terephthalate and so forth; polyvinyl acetate; polyvinyl chloride acetate; polyvinyl butyral; acrylic resins, e.g., polyacrylate, polymethylacrylate, polymethylmethacrylate, and so forth; polyamides, e.g., nylon; polyvinyl chloride; polyvinylidene chloride; polystyrene; polyvinyl alcohol; polyurethanes; polylactic acid; copolymers thereof; blends thereof; and so forth. It should be noted that the polymer(s) may also contain other additives, such as processing aids or treatment compositions to impart desired properties to the fibers, residual amounts of solvents, pigments or colorants, and so forth.

Monocomponent and/or multicomponent fibers may be used to form the nonwoven facing. Monocomponent fibers are generally formed from a polymer or blend of polymers extruded from a single extruder. Multicomponent fibers are generally formed from two or more polymers (e.g., bicomponent fibers) extruded from separate extruders. The polymers may be arranged in substantially constantly positioned distinct zones across the cross-section of the fibers. The components may be arranged in any desired configuration, such as sheath-core, side-by-side, pie, island-in-the-sea, three island, bull's eye, or various other arrangements known in the art. Various methods for forming multicomponent fibers are described in U.S. Pat. No. 4,789,592 to Taniguchi et al. and U.S. Pat. Nos. 5,336,552 to Strack et al., 5,108,820 to Kaneko, et al., 4,795,668 to Kruege, et al., 5,382,400 to Pike, et al., 5,336,552 to Strack, et al., and 6,200,669 to Marmon, et al., which are incorporated herein in their entirety by reference thereto for all purposes. Multicomponent fibers having various irregular shapes may also be formed, such as described in U.S. Pat. Nos. 5,277,976 to Hogle, et al., 5,162,074 to Hills, 5,466,410 to Hills, 5,069,970 to Largman, et al., and 5,057,368 to Largman, et al., which are incorporated herein in their entirety by reference thereto for all purposes.

The desired denier of the fibers used to form the nonwoven facing may vary depending on the desired application. Typically, the fibers are formed to have a denier per filament (i.e., the unit of linear density equal to the mass in grams per 9000 meters of fiber) of less than about 6, in further embodiments less than about 3, and in even further embodiments, from about 0.5 to about 3.

Although not required, the nonwoven facing may be optionally bonded using any conventional technique, such as with an adhesive or autogenously (e.g., fusion and/or self-adhesion of the fibers without an applied external adhesive). Suitable autogenous bonding techniques may include ultrasonic bonding, thermal bonding, through-air bonding, calender bonding, and so forth. The temperature and pressure required may vary depending upon many factors including but not limited to, pattern bond area, polymer properties, fiber properties and nonwoven properties. For example, the facing may be passed through a nip formed between two rolls, both of which are typically not patterned i.e., smooth. In this manner, only a small amount of pressure is exerted on the materials to lightly bond them together. Without intending to be limited by theory, the present inventors believe that such lightly bonded materials can retain a higher degree of extensibility and thereby increase the elasticity and extensibility of the resulting composite. For example, the nip pressure may range from about 0.1 to about 20 pounds per linear inch, in further embodiments from about 1 to about 15 pounds per linear inch, and in even further embodiments, from about 2 to about 10 pounds per linear inch. One or more of the rolls may likewise have a surface temperature of from about 15° C. to about 60° C., in further embodiments from about 20° C. to about 50° C., and in even further embodiments, from about 25° C. to about 40° C.

The nonwoven facing may also be stretched in the machine and/or cross-machine directions prior to lamination to the film of the present invention. Suitable stretching techniques may include necking, tentering, groove roll stretching, etc. For example, the facing may be necked such as described in U.S. Pat. Nos. 5,336,545, 5,226,992, 4,981,747 and 4,965,122 to Morman, as well as U.S. Patent Application Publication No. 2004/0121687 to Morman, et al. Alternatively, the nonwoven facing may remain relatively inextensible in at least one direction prior to lamination to the film. In such embodiments, the nonwoven facing may be optionally stretched in one or more directions subsequent to lamination to the film. The facing may also be subjected to other known processing steps, such as aperturing, heat treatments, etc.

### II. Elastic Film

The elastic film of the present invention is formed from one or more elastomeric polymers that are melt-processable, i.e., thermoplastic. Any of a variety of thermoplastic elastomeric polymers may generally be employed in the present invention, such as elastomeric polyesters, elastomeric polyurethanes, elastomeric polyamides, elastomeric copolymers, elastomeric polyolefins, and so forth. In one embodiment, for instance, a substantially amorphous block copolymer may be employed that contains blocks of a monoalkenyl arene and a saturated conjugated diene. Such block copolymers are particularly useful in the present invention due to their high degree of elasticity and tackiness, which enhances the ability of the film to bond to the nonwoven facing.

The monoalkenyl arene block(s) may include styrene and its analogues and homologues, such as o-methyl styrene; p-methyl styrene; p-tert-butyl styrene; 1,3 dimethyl styrene p-methyl styrene; etc., as well as other monoalkenyl polycyclic aromatic compounds, such as vinyl naphthalene; vinyl anthrycene; and so forth. Preferred monoalkenyl arenes are styrene and p-methyl styrene. The conjugated diene block(s) may include homopolymers of conjugated diene monomers, copolymers of two or more conjugated dienes, and copolymers of one or more of the dienes with another monomer in which the blocks are predominantly conjugated diene units. Preferably, the conjugated dienes contain from 4 to 8 carbon atoms, such as 1,3 butadiene (butadiene); 2-methyl-1,3 butadiene; isoprene; 2,3 dimethyl-1,3 butadiene; 1,3 pentadiene (piperylene); 1,3 hexadiene; and so forth. The amount of monoalkenyl arene (e.g., polystyrene) blocks may vary, but typically constitute from about 8 wt % to about 55 wt %, in further embodiments from about 10 wt % to about 35 wt %, and in even further embodiments, from about 25 wt % to about 35 wt % of the copolymer. Suitable block copolymers may contain monoalkenyl arene endblocks having a number average molecular weight from about 5,000 to about 35,000 and saturated conjugated diene midblocks having a number average molecular weight from about 20,000 to about 170,000. The total number average molecular weight of the block polymer may be from about 30,000 to about 250,000.

Particularly suitable thermoplastic elastomeric copolymers are available from Kraton Polymers LLC of Houston, Tex. under the trade name KRATON®. KRATON® polymers include styrene-diene block copolymers, such as styrene-butadiene, styrene-isoprene, styrene-butadiene-styrene, and styrene-isoprene-styrene. KRATON® polymers also include styrene-olefin block copolymers formed by selective hydrogenation of styrene-diene block copolymers. Examples of such styrene-olefin block copolymers include styrene-(ethylene-butylene), styrene-(ethylene-propylene), styrene-(ethylene-butylene)-styrene, styrene-(ethylene-propylene)-styrene, styrene-(ethylene-butylene)-styrene-(ethylene-butylene), styrene-(ethylene-propylene)-styrene-(ethylene-propylene), and styrene-ethylene-(ethylene-propylene)-styrene. These block copolymers may have a linear, radial or star-shaped molecular configuration. Specific KRATON® block copolymers include those sold under the brand names G 1652, G 1657, G 1730, MD6673, and MD6973. Various suitable styrenic block copolymers are described in U.S. Pat. Nos. 4,663,220, 4,323,534, 4,834,738, 5,093,422 and 5,304,599, which are hereby incorporated in their entirety by reference thereto for all purposes. Other commercially available block copolymers include the S-EP-S elastomeric copolymers available from Kuraray Company, Ltd. of Okayama, Japan, under the trade designation SEPTON®. Still other suitable copolymers include the S-I-S and S-B-S elastomeric copolymers available from Dexco Polymers of Houston, Tex. under the trade designation VECTOR®. Also suitable are polymers composed of an A-B-A-B tetrablock copolymer, such as discussed in U.S. Pat. No. 5,332,613 to Taylor, et al., which is incorporated herein in its entirety by reference thereto for all purposes. An example of such a tetrablock copolymer is a styrene-poly(ethylene-propylene)-styrene-poly(ethylene-propylene) (“S-EP-S-EP”) block copolymer.

Of course, other thermoplastic elastomeric polymers may also be used to form the film, either alone or in conjunction with the block copolymers. Semi-crystalline polyolefins, for example, may be employed that have or are capable of exhibiting a substantially regular structure. Exemplary semi-crystalline polyolefins include polyethylene, polypropylene, blends and copolymers thereof. In one particular embodiment, a polyethylene is employed that is a copolymer of ethylene and an α-olefin, such as a C₃-C₂₀ α-olefin or C₃-C₁₂ α-olefin. Suitable α-olefins may be linear or branched (e.g., one or more C₁-C₃ alkyl branches, or an aryl group). Specific examples include 1-butene; 3-methyl-1-butene; 3,3-dimethyl-1-butene; 1-pentene; 1-pentene with one or more methyl, ethyl or propyl substituents; 1-hexene with one or more methyl, ethyl or propyl substituents; 1-heptene with one or more methyl, ethyl or propyl substituents; 1-octene with one or more methyl, ethyl or propyl substituents; 1-nonene with one or more methyl, ethyl or propyl substituents; ethyl, methyl or dimethyl-substituted 1-decene; 1-dodecene; and styrene. Particularly desired α-olefin comonomers are 1-butene, 1-hexene and 1-octene. The ethylene content of such copolymers may be from about 60 mole % to about 99 mole %, in further embodiments from about 80 mole % to about 98.5 mole %, and in even further embodiments, from about 87 mole % to about 97.5 mole %. The α-olefin content may likewise range from about 1 mole % to about 40 mole %, in further embodiments from about 1.5 mole % to about 15 mole %, and in even further embodiments, from about 2.5 mole % to about 13 mole %.

Particularly suitable polyethylene copolymers are those that are “linear” or “substantially linear.” The term “substantially linear” means that, in addition to the short chain branches attributable to comonomer incorporation, the ethylene polymer also contains long chain branches in that the polymer backbone. “Long chain branching” refers to a chain length of at least 6 carbons. Each long chain branch may have the same comonomer distribution as the polymer backbone and be as long as the polymer backbone to which it is attached. Suitable substantially linear polymers are substituted with from 0.01 long chain branch per 1000 carbons to 1 long chain branch per 1000 carbons, and in further embodiments, from 0.05 long chain branch per 1000 carbons to 1 long chain branch per 1000 carbons. In contrast to the term “substantially linear”, the term “linear” means that the polymer lacks measurable or demonstrable long chain branches. That is, the polymer is substituted with an average of less than 0.01 long chain branch per 1000 carbons.

The density of a linear ethylene/α-olefin copolymer is a function of both the length and amount of the α-olefin. That is, the greater the length of the α-olefin and the greater the amount of α-olefin present, the lower the density of the copolymer.

Although not necessarily required, linear polyethylene “plastomers” are particularly desirable in that the content of α-olefin short chain branching content is such that the ethylene copolymer exhibits both plastic and elastomeric characteristics—i.e., a “plastomer.” Because polymerization with α-olefin comonomers decreases crystallinity and density, the resulting plastomer normally has a density lower than that of polyethylene thermoplastic polymers (e.g., LLDPE), but approaching and/or overlapping that of an elastomer. For example, the density of the polyethylene plastomer may be 0.91 grams per cubic centimeter (g/cm³) or less, in further embodiments, from 0.85 to 0.88 g/cm³, and in even further embodiments, from 0.85 g/cm³ to 0.87 g/cm³. Despite having a density similar to elastomers, plastomers generally exhibit a higher degree of crystallinity, are relatively non-tacky, and may be formed into pellets that are non-adhesive and relatively free flowing.

Suitable polyethylenes for use in the present invention are ethylene-based copolymer plastomers available under the designation EXACT™ from ExxonMobil Chemical Company of Houston, Tex. Other suitable polyethylene plastomers are available under the designation ENGAGE™ and AFFINITY™ from Dow Chemical Company of Midland, Mich. Still other suitable ethylene polymers are available from The Dow Chemical Company under the designations DOWLEX™ (LLDPE) and ATTANE™ (ULDPE). Other suitable ethylene polymers are described in U.S. Pat. Nos. 4,937,299 to Ewen et al.; 5,218,071 to Tsutsui et al.; 5,272,236 to Lai, et al.; and 5,278,272 to Lai, et al., which are incorporated herein in their entirety by reference thereto for all purposes.

Of course, the present invention is by no means limited to the use of ethylene polymers. For instance, propylene plastomers may also be suitable for use in the film. Suitable plastomeric propylene polymers may include, for instance, copolymers or terpolymers of propylene include copolymers of propylene with an α-olefin (e.g., C₃-C₂₀), such as ethylene, 1-butene, 2-butene, the various pentene isomers, 1-hexene, 1-octene, 1-nonene, 1-decene, 1-unidecene, 1-dodecene, 4-methyl-1-pentene, 4-methyl-1-hexene, 5-methyl-1-hexene, vinylcyclohexene, styrene, etc. The comonomer content of the propylene polymer may be about 35 wt % or less, in further embodiments from about 1 wt % to about 20 wt %, and in even further embodiments, from about 2 wt % to about 10 wt %. Suitably, the density of the polypropylene (e.g., propylene/α-olefin copolymer) may be 0.91 grams per cubic centimeter (g/cm³) or less, in further embodiments, from 0.85 to 0.88 g/cm³, and in even further embodiments, from 0.85 g/cm³ to 0.87 g/cm³. Suitable propylene polymers are commercially available under the designations VISTAMAXX™ from ExxonMobil Chemical Co. of Houston, Tex.; TAFMER™ available from Mitsui Petrochemical Industries; and VERSIFY™ available from The Dow Chemical Company of Midland, Mich. Other examples of suitable propylene polymers are described in U.S. Pat. No. 6,500,563 to Datta, et al.; U.S. Pat. No. 5,539,056 to Yang, et al.; and U.S. Pat. No. 5,596,052 to Resconi, et al., which are incorporated herein in their entirety by reference thereto for all purposes.

Any of a variety of known techniques may generally be employed to form the semi-crystalline polyolefins. For instance, olefin polymers may be formed using a free radical or a coordination catalyst (e.g., Ziegler-Natta). Preferably, the olefin polymer is formed from a single-site coordination catalyst, such as a metallocene catalyst. Such a catalyst system produces ethylene copolymers in which the comonomer is randomly distributed within a molecular chain and uniformly distributed across the different molecular weight fractions. Metallocene-catalyzed polyolefins are described, for instance, in U.S. Pat. Nos. 5,571,619 to McAlpin et al.; 5,322,728 to Davis et al.; 5,472,775 to Obijeski et al.; 5,272,236 to Lai et al.; and 6,090,325 to Wheat, et al., which are incorporated herein in their entirety by reference thereto for all purposes. Examples of metallocene catalysts include bis(n-butylcyclopentadienyl)titanium dichloride, bis(n-butylcyclopentadienyl)zirconium dichloride, bis(cyclopentadienyl)scandium chloride, bis(indenyl)zirconium dichloride, bis(methylcyclopentadienyl)titanium dichloride, bis(methylcyclopentadienyl)zirconium dichloride, cobaltocene, cyclopentadienyltitanium trichloride, ferrocene, hafnocene dichloride, isopropyl(cyclopentadienyl,-1-flourenyl)zirconium dichloride, molybdocene dichloride, nickelocene, niobocene dichloride, ruthenocene, titanocene dichloride, zirconocene chloride hydride, zirconocene dichloride, and so forth. Polymers made using metallocene catalysts typically have a narrow molecular weight range. For instance, metallocene-catalyzed polymers may have polydispersity numbers (M_{w}/Mₙ) of below 4, controlled short chain branching distribution, and controlled isotacticity.

The melt flow index (MI) of the semi-crystalline polyolefins may generally vary, but is typically in the range of about 0.1 grams per 10 minutes to about 100 grams per 10 minutes, in further embodiments from about 0.5 grams per 10 minutes to about 30 grams per 10 minutes, and in even further embodiments, about 1 to about 10 grams per 10 minutes, determined at 190° C. The melt flow index is the weight of the polymer (in grams) that may be forced through an extrusion rheometer orifice (0.0825-inch diameter) when subjected to a force of 5000 grams in 10 minutes at 190° C., and may be determined in accordance with ASTM Test Method D1238-E.

Of course, besides elastomeric polymers, generally inelastic thermoplastic polymers may also be used so long as they do not adversely affect the elasticity of the composite. For example, the thermoplastic composition may contain other polyolefins (e.g., polypropylene, polyethylene, etc.). In one embodiment, the thermoplastic composition may contain an additional propylene polymer, such as homopolypropylene or a copolymer of propylene. The additional propylene polymer may, for instance, be formed from a substantially isotactic polypropylene homopolymer or a copolymer containing equal to or less than about 10 wt % of other monomer, i.e., at least about 90% by weight propylene. Such a polypropylene may be present in the form of a graft, random, or block copolymer and may be predominantly crystalline in that it has a sharp melting point above about 110° C., in further embodiments about above 115° C., and in even further embodiments, above about 130° C. Examples of such additional polypropylenes are described in U.S. Pat. No. 6,992,159 to Datta, et al., which is incorporated herein in its entirety by reference thereto for all purposes.

The elastic film of the present invention may also contain other components as is known in the art. In one embodiment, for example, the elastic film contains a filler. Fillers are particulates or other forms of material that may be added to the film polymer extrusion blend and that will not chemically interfere with the extruded film, but which may be uniformly dispersed throughout the film. Fillers may serve a variety of purposes, including enhancing film opacity and/or breathability (i.e., vapor-permeable and substantially liquid-impermeable). For instance, filled films may be made breathable by stretching, which causes the polymer to break away from the filler and create microporous passageways. Breathable microporous elastic films are described, for example, in U.S. Pat. Nos. 5,997,981; 6,015,764; and 6,111,163 to McCormack, et al.; 5,932,497 to Morman, et al.; 6,461,457 to Taylor, et al., which are incorporated herein in their entirety by reference thereto for all purposes. Examples of suitable fillers include, but are not limited to, calcium carbonate, various kinds of clay, silica, alumina, barium carbonate, sodium carbonate, magnesium carbonate, talc, barium sulfate, magnesium sulfate, aluminum sulfate, titanium dioxide, zeolites, cellulose-type powders, kaolin, mica, carbon, calcium oxide, magnesium oxide, aluminum hydroxide, pulp powder, wood powder, cellulose derivatives, chitin and chitin derivatives. In certain cases, the filler content of the film may range from about 25 wt % to about 75 wt %, in further embodiments, from about 30 wt % to about 70 wt %, and in even further embodiments, from about 40 wt % to about 60 wt % of the film.

Other additives may also be incorporated into the film, such as melt stabilizers, crosslinking catalysts, pro-rad additives, processing stabilizers, heat stabilizers, light stabilizers, antioxidants, heat aging stabilizers, whitening agents, antiblocking agents, bonding agents, tackifiers, viscosity modifiers, etc. Examples of suitable tackifier resins may include, for instance, hydrogenated hydrocarbon resins. REGALREZ™ hydrocarbon resins are examples of such hydrogenated hydrocarbon resins, and are available from Eastman Chemical. Other tackifiers are available from ExxonMobil under the ESCOREZ™ designation. Viscosity modifiers may also be employed, such as polyethylene wax (e.g., EPOLENE™ C-10 from Eastman Chemical). Phosphite stabilizers (e.g., IRGAFOS available from Ciba Specialty Chemicals of Terrytown, N.Y. and DOVERPHOS available from Dover Chemical Corp. of Dover, Ohio) are exemplary melt stabilizers. In addition, hindered amine stabilizers (e.g., CHIMASSORB available from Ciba Specialty Chemicals) are exemplary heat and light stabilizers. Further, hindered phenols are commonly used as an antioxidant in the production of films. Some suitable hindered phenols include those available from Ciba Specialty Chemicals of under the trade name “Irganox®”, such as Irganox® 1076, 1010, or E 201. Moreover, bonding agents may also be added to the film to facilitate bonding of the film to additional materials (e.g., nonwoven web). Typically, such additives (e.g., tackifier, antioxidant, stabilizer, etc.) are each present in an amount from about 0.001 wt % to about 25 wt %, in further embodiments, from about 0.005 wt % to about 20 wt %, and in even further embodiments, from 0.01 wt % to about 15 wt % of the film.

The elastic film of the present invention may be mono- or multi-layered. Multi-layered films may be prepared by co-extrusion or any other conventional layering technique. When employed, the multi-layered film typically contains at least one thermoplastic layer and at least one elastic layer. The thermoplastic layer may be employed to provide strength and integrity to the resulting composite, while the elastic layer may be employed to provide elasticity and sufficient tack for adhering to the nonwoven facing. In one particular embodiment of the present invention, the film includes at least one thermoplastic layer positioned between at least two elastic layers. In this manner, the thermoplastic layer does not substantially contact the nonwoven facing and is thus able to avoid substantial damage during lamination. In such embodiments, one or more elastic layers are generally formed from an elastomeric composition, such as described above, to provide the desired degree of elasticity in the film. To impart the desired elastic properties to the film, elastomers typically constitute about 55 wt % or more, in further embodiments about 60 wt % or more, and in even further embodiments, from about 65 wt % to 100 wt % of the polymer content of the elastomeric composition used to form the elastic layer(s). In fact, in certain embodiments, the elastic layer(s) may be generally free of polymers that are inelastic. For example, such inelastic polymers may constitute about 15 wt % or less, in further embodiments about 10 wt % or less, and in even further embodiments, about 5 wt % or less of the polymer content of the elastomeric composition.

Although the thermoplastic layer(s) may possess some degree of elasticity, such layers are generally formed from a thermoplastic composition that is less elastic than the elastic layer(s) to ensure that the strength of the film is sufficiently enhanced. For example, one or more elastic layers may be formed primarily from substantially amorphous elastomers (e.g., styrene-olefin copolymers) and one or more thermoplastic layers may be formed from polyolefin plastomers (e.g., single-site catalyzed ethylene or propylene copolymers), which are described in more detail above. Although possessing some elasticity, such polyolefins are generally less elastic than substantially amorphous elastomers. Of course, the thermoplastic layer(s) may contain generally inelastic polymers, such as conventional polyolefins, e.g., polyethylene (low density polyethylene (“LDPE”), Ziegler-Natta catalyzed linear low density polyethylene (“LLDPE”), etc.), polypropylene, polybutylene, etc.; polytetrafluoroethylene; polyesters, e.g., polyethylene terephthalate, etc.; polyvinyl acetate; polyvinyl chloride acetate; polyvinyl butyral; acrylic resins, e.g., polyacrylate, polymethylacrylate, polymethylmethacrylate, etc.; polyamides, e.g., nylon; polyvinyl chloride; polyvinylidene chloride; polystyrene; polyvinyl alcohol; polyurethanes; polylactic acid; copolymers and mixtures thereof; and so forth. In certain embodiments, polyolefins (e.g., conventional and/or plastomers) are employed and constitute about 55 wt % or more, in further embodiments about 60 wt % or more, and in even further embodiments, from about 65 wt % to 100 wt % of the polymer content of the thermoplastic composition used to form the thermoplastic layer(s).

The thickness of the thermoplastic and elastic layers is generally selected so as to achieve an appropriate balance between film elasticity and strength. For instance, the thickness of an elastic layer is typically from about 20 to about 200 micrometers, in further embodiments from about 25 to about 175 micrometers, and in even further embodiments, from about 30 to about 150 micrometers. The elastic layer(s) may also constitute from about 70% to about 99.5% of the total thickness of the film, and in further embodiments from about 80% to about 99% of the total thickness of the film. On the other hand, the thickness of a thermoplastic layer(s) is typically from about 0.5 to about 20 micrometers, in further embodiments from about 1 to about 15 micrometers, and in even further embodiments, from about 2 to about 12 micrometers. The thermoplastic layer(s) may also constitute from about 0.5% to about 30% of the total thickness of the film, and in further embodiments from about 1% to about 20% of the total thickness of the film. The film may also have a total thickness of from about 20 to about 250 micrometers, in further embodiments, from about 25 to about 225 micrometers, and in even further embodiments, from about 30 to about 200 micrometers.

Regardless of the particular film content, the film and/or the materials used to form the film may also be subjected to one or more additional processing steps. In one embodiment, for example, an elastomeric polymer employed in the film is crosslinked, before, after, and/or during lamination to the nonwoven facing, to provide the film with enhanced elastic characteristics. Crosslinking may be induced by subjecting the polymer to electromagnetic radiation, such as ultraviolet light, electron beam radiation, natural and artificial radio isotopes (e.g., α, β, and γ rays), x-rays, neutron beams, positively-charged beams, laser beams, and so forth. The wavelength (“λ”) of the electromagnetic radiation may be about 1000 nanometers or less, in further embodiments about 100 nanometers or less, and in even further embodiments, about 1 nanometer or less. Electron beam radiation, for instance, typically has a wavelength of about 1 nanometer or less. The total dosage employed (in one or multiple steps) may likewise range from about 1 megarad (Mrad) to about 30 Mrads, in further embodiments, from about 3 Mrads to about 25 Mrads, and in even further embodiments, from about 5 to about 15 Mrads. In addition, the energy level may range from about 0.05 megaelectron volts (MeV) to about 600 MeV. Upon crosslinking, a three-dimensional crosslinked network may be formed that provides the material with additional elasticity in the machine direction, cross-machine direction, or both.

### III. Other Facings

If desired, the composite of the present invention may also include other facings as is known in the art, such as nonwoven web materials, films, foams, etc. For example, the composite may include an additional nonwoven facing, such as a meltblown web, spunbond web, bonded carded web, wetlaid web, airlaid web, coform web, etc., as well as combinations of the foregoing. In one particular embodiment, the additional facing may be a bonded carded facing. Fibers of any desired length may be employed in the bonded carding facing, such as staple fibers, continuous fibers, etc. For example, staple fibers may be used that have a fiber length in the range of from about 1 to about 150 millimeters, in further embodiments from about 5 to about 50 millimeters, in even further embodiments from about 10 to about 40 millimeters, and in even further embodiments, from about 10 to about 25 millimeters. Such fibers may be formed into a carded web by placing bales of the fibers into a picker that separates the fibers. Next, the fibers are sent through a combing or carding unit that further breaks apart and aligns the fibers in the machine direction so as to form a machine direction-oriented fibrous nonwoven web. The carded web may then be lightly bonded in a manner such as described above.

Although not required, the additional facing may also be lightweight and of low strength. For example, the basis weight of the facing may range from about 1 to about 45 grams per square meter, in further embodiments from about 2 to about 30 grams per square meter, and in even further embodiments, from about 3 to about 20 grams per square meter. The facing may also have a peak load in the cross-machine direction (“CD”) of about 350 grams-force per inch (width) or less, in further embodiments about 300 grams-force per inch or less, in even further embodiments from about 50 to about 300 grams-force per inch, in even further embodiments from about 60 to about 250 grams-force per inch, and in even further embodiments, from about 75 to about 200 grams-force per inch. If desired, the nonwoven facing may also have a low strength in the machine direction (“MD”), such as a peak load in the machine direction of about 3000 grams-force per inch (width) or less, in further embodiments about 2500 grams-force per inch or less, in even further embodiments from about 50 to about 2000 grams-force per inch, and in even further embodiments, from about 100 to about 1500 grams-force per inch.

As described above, the additional nonwoven facing may also be stretched in the machine and/or cross-machine directions prior to lamination to the film of the present invention, as well as subjected to other known processing steps, such as aperturing, heat treatments, etc.

### IV. Lamination Technique

To enhance the durability and stability of the resulting composite, the film is typically laminated to the facing by directly extruding the elastomeric composition onto a surface of the nonwoven facing. This allows for an enhanced degree of contact between the elastomeric composition and fibers of the nonwoven facing, which further increases the ability of the meltblown fibers to bond to the elastomeric composition. In this manner, a sufficient degree of bonding is achieved without requiring the application of a substantial amount of heat and pressure used in conventional calender bonding processes, which can damage the low strength nonwoven facing. If desired, lamination may be facilitated through the use of a variety of techniques, such as adhesives, suctional forces, etc. In one embodiment, for example, the film is biased toward the facing during lamination with a suctional force.

Regardless of the lamination technique employed, the selection of an appropriate bonding temperature will help melt and/soften the elastomeric polymer(s) of the film so that it may flow and become fused to the nonwoven facing, thereby forming an integral nonwoven composite. Furthermore, because the elastomeric polymer(s) may physically entrap or adhere to the fibers at the bond sites, adequate bond formation may be achieved without requiring substantial softening of the polymer(s) used to form the nonwoven facing. Of course, it should be understood that the temperature of the nonwoven facing may be above its softening point in certain embodiments. To achieve the desired degree of bond formation between the film and nonwoven facing, the temperature at which the elastomeric composition is extruded is typically from about 50° C. to about 300° C., in further embodiments from about 60° C. to about 275° C., and in even further embodiments, from about 70° C. to about 260° C.

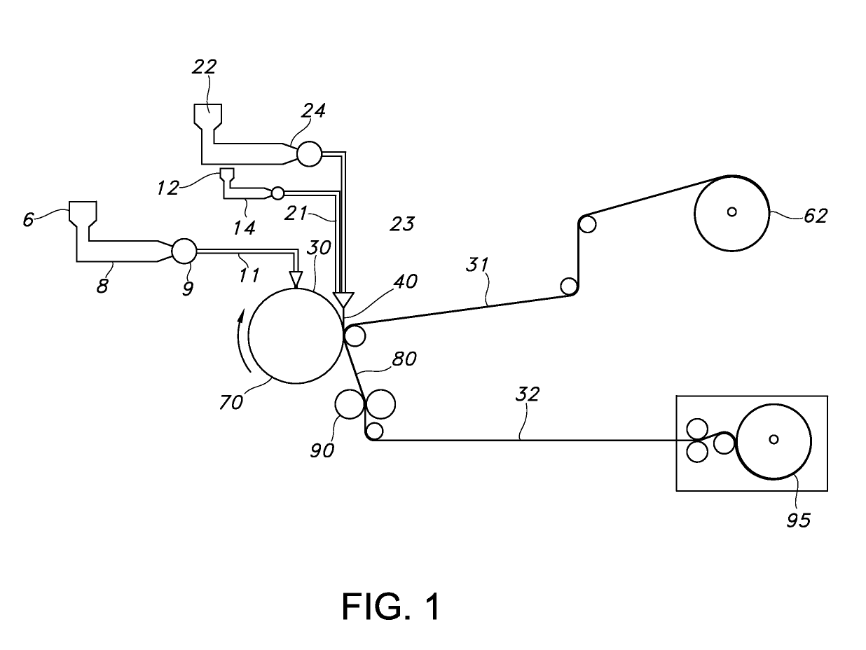

Various embodiments of the lamination technique of the present invention will now be described in greater detail. Referring to FIG. 1, for instance, one embodiment of a method for forming a composite from an elastic film and a meltblown facing is shown. In this embodiment, a meltblown facing 30 is formed in-line by feeding raw materials (e.g., polypropylene) into an extruder 8 from a hopper 6, and thereafter supplying the extruded composition to a meltblown die 9. As the polymer exits the die 9 at an orifice (not shown), high pressure fluid (e.g., heated air) attenuates and spreads the polymer stream into microfibers 11 that are randomly deposited onto a surface of a roll 70 to form a meltblown facing 30. It should be understood that the meltblown facing 30 may also be formed on a separate foraminous surface (e.g., wire, belt, fabric, etc.) that subsequently traverses over the roll 70. Further, it should be understood that the meltblown facing 30 may simply be unwound from a supply roll rather than formed in-line

In the embodiment shown in FIG. 1, an elastic film is also formed that contains a single thermoplastic layer 23 and a single elastic layer 21. More specifically, the raw materials of the elastic layer 21 may be added to a hopper 12 of an extruder 14 and the raw materials of the thermoplastic layer 23 may be added to a hopper 22 of an extruder 24. The materials are dispersively mixed and compounded under at an elevated temperature within the extruders 14 and 24. Within the extruder 14, for example, melt blending of the elastomeric composition may occur at a temperature of from about 50° C. to about 300° C., in further embodiments from about 60° C. to about 275° C., and in even further embodiments, from about 70° C. to about 260° C. Melt blending of the thermoplastic composition may occur within the extruder 24 at a temperature that is the same, lower, or higher than employed for the elastomeric composition. For example, melt blending of the thermoplastic composition may occur in some instances at a temperature of from about 50° C. to about 250° C., in further embodiments from about 60° C. to about 225° C., and in even further embodiments, from about 70° C. to about 200° C. The apparent shear rate during melt blending may range from about 100 seconds⁻¹ to about 10,000 seconds⁻¹, in further embodiments from about 500 seconds⁻¹ to about 5000 seconds⁻¹, and in even further embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows.

Any known technique may be used to form a film from the compounded material, including casting, flat die extruding, etc. In the particular embodiment of FIG. 1, for example, the elastic and thermoplastic layers are “cast” onto the meltblown facing 30, which is positioned on the roll 70, as is known in the art. A cast film 40 is thus formed on the facing 30 such that the elastic layer 21 is positioned directly adjacent to the facing 30. To enhance bonding between the film 40 and the facing 30, a suctional force may be applied to bias the film 40 against an upper surface of the meltblown facing 30. This may be accomplished in a variety of ways (e.g., vacuum slots, shoes, rolls, etc.) and at a variety of locations throughout the composite-forming process. In the embodiment shown in FIG. 1, for example, the roll 70 on which the film 40 is cast is a vacuum roll capable of applying the desired suctional force. The amount of suctional force may be selectively controlled to enhance bonding without significantly deteriorating the integrity of the low strength facing. For example, pneumatic vacuum pressure may be employed to apply the suctional force that is about 0.25 kilopascals or more, in further embodiments about from about 0.3 to about 5 kilopascals, and in even further embodiments, from about 0.5 to about 2 kilopascals. Such vacuum-assisted lamination allows for the formation of a strong composite without the need for a substantial amount of heat and pressure normally used in calender lamination methods that could otherwise diminish the integrity of the nonwoven facing. In fact, the roll 70 on which the film 40 is formed may even be kept at ambient temperature if so desired.

Although not necessarily required, a second facing 31 may also be laminated to the elastic film 40. The second facing 31 may be formed in-line or originated from a supply roll (e.g., roll 62). The second facing 31 may be a nonwoven facing, as well as another type of nonwoven web material, film, foam, etc. Upon lamination, the elastic film 40 is melt fused to the facings 30 and 31 at a plurality of discrete bond sites to form a composite 80. That is, the elastomeric polymer(s) of the film 40 are softened and/or melted so that they may physically entrap fibers of the materials 30 and 31. The elastic film 40 may possess a certain tack so that it also adheres to the fibers upon lamination. If desired, bonding may occur at a temperature that is insufficient to substantially soften the polymer(s) of the facings 30 and 31 so that they are not substantially melt fused to each other. In this manner, the resulting composite 80 may better retain the physical properties (e.g., liquid permeability, softness, bulk, and hand feel) of the nonwoven facings.

### V. Aperturing Technique

To form apertures in the film-nonwoven laminate, the aperturing is generally accomplished in the present invention via feeding the film/nonwoven laminate through a nip defined by at least one patterned roll. The patterned roll contains a plurality of raised elements to form the apertures in the film-nonwoven laminate. The size of the raised elements may be specifically tailored to facilitate the formation of apertures extending through the thickness of the film-nonwoven laminate. For example, the raised elements are typically selected to have a relatively large length dimension. The length dimension of the raised elements may be from about 300 to about 5000 micrometers, in further embodiments from about 500 to about 4000 micrometers, and in even further embodiments, from about 1000 to about 2000 micrometers. The width dimension of the raised elements may likewise range from about 20 to about 500 micrometers, in further embodiments from about 40 to about 200 micrometers, and in even further embodiments, from about 50 to about 150 micrometers. In addition, the “element aspect ratio” (the ratio of the length of an element to its width) may range from about 2 to about 100, in further embodiments from about 4 to about 50, and in even further embodiments, from about 5 to about 20.

Besides the size of the raised elements, the overall element pattern may also be selectively controlled to achieve the desired aperture formation. In one embodiment, for example, a pattern is selected in which the longitudinal axis (longest dimension along a center line of the element) of one or more of the raised elements is skewed relative to the machine direction (“MD”) of the film-nonwoven laminate. For example, one or more of the raised elements may be oriented from about 30° to about 150°, in further embodiments from about 45° to about 135°, and in even further embodiments, from about 60° to about 120° relative to the machine direction of the film-nonwoven laminate. In this manner, the raised elements will present a relatively large surface to the film-nonwoven laminate in a direction substantially perpendicular to that which the film-nonwoven laminate moves. This increases the area over which shear stress is imparted to the film-nonwoven laminate and, in turn, facilitates aperture formation.

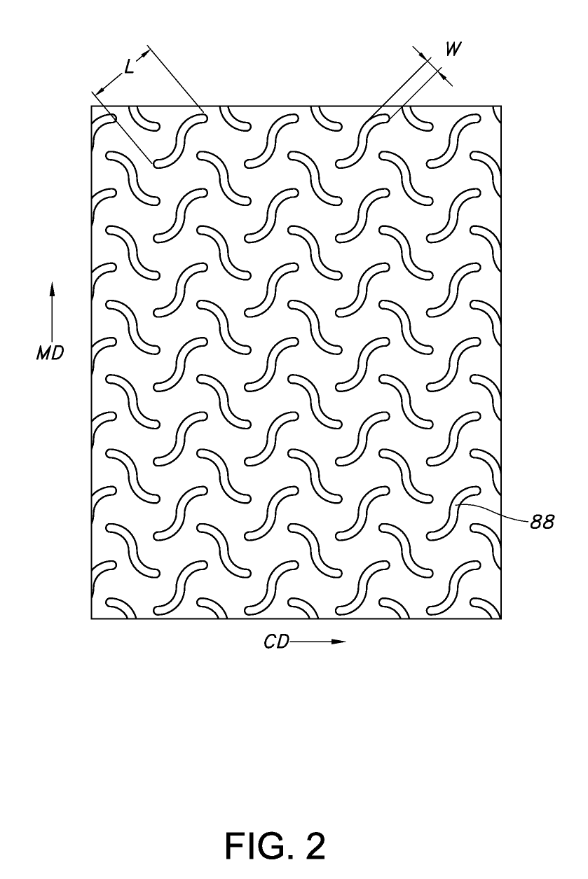

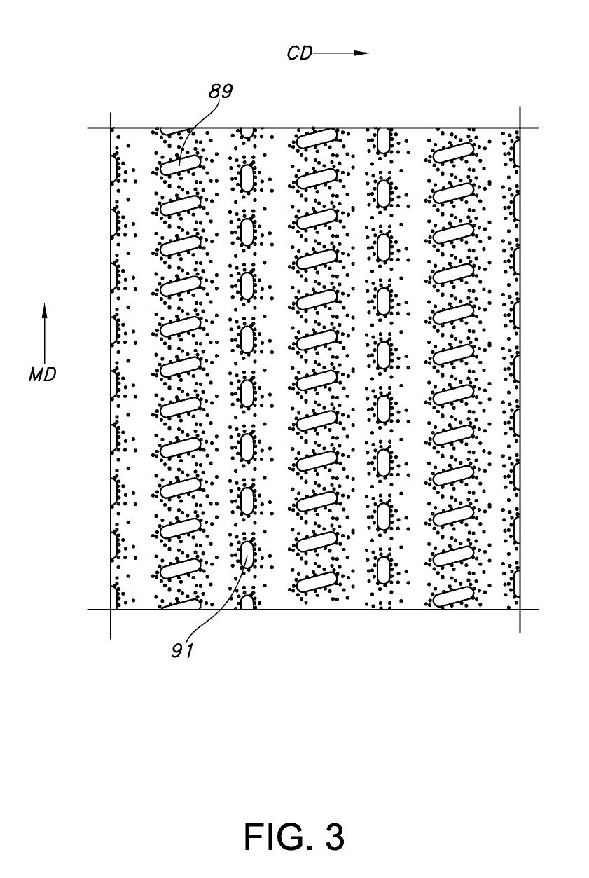

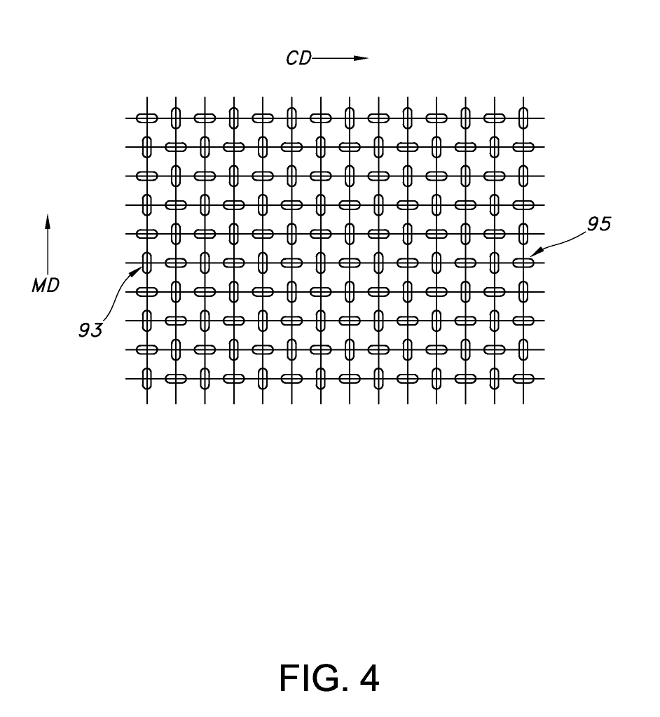

The pattern of the raised elements is generally selected so that the film-nonwoven laminate has a total apertured area and/or embossed area of less than about 50% (as determined by conventional optical microscopic methods), and in further embodiments, less than about 30%. The density of the pattern is also typically greater than about 10 raised elements (apertures) per square inch, and in further embodiments, from about 20 to about 500 raised elements per square inch. One suitable pattern of raised elements is known as an “S-weave” pattern and is described in U.S. Pat. No. 5,964,742 to McCormack, et al., which is incorporated herein in its entirety by reference thereto for all purposes. S-weave patterns typically have a raised element density of from about 50 to about 500 raised elements per square inch, and in further embodiments, from about 75 to about 150 raised elements per square inch. An example of a suitable “S-weave” pattern in shown in FIG. 2, which illustrates S-shaped raised elements 88 having a length dimension “L” and a width dimension “W.” Another suitable element pattern is known as the “rib-knit” pattern and is described in U.S. Pat. No. 5,620,779 to Levy, et al., which is incorporated herein in its entirety by reference thereto for all purposes. Rib-knit patterns typically have a raised element density of from about 150 to about 400 raised elements per square inch, and in further embodiments, from about 200 to about 300 raised elements per square inch. An example of a suitable “rib-knit” pattern in shown in FIG. 3, which illustrates first raised elements 89 and second raised elements 91, which second raised elements are oriented in a different direction than the first raised elements. Yet another suitable pattern is the “wire weave” pattern, which has a raised element density of from about 200 to about 500 raised elements per square inch, and in further embodiments, from about 250 to about 350 raised elements per square inch. An example of a suitable “wire-weave” pattern in shown in FIG. 4, which illustrates first raised elements 93 and second raised elements 95, which second raised elements are oriented in a different direction than the first raised elements. Another suitable element pattern may have diamond-shaped pins that have a pin density of 53 pins per square inch and cover approximately 8.1 percent of the area on the surface of the roll. An even further suitable element pattern may have round elements with a density of 32 elements per square inch and a cover approximately 9.8% of the area on the surface of the roll. Other bond patterns that may be used in the present invention are described in U.S. Pat. Nos. 3,855,046 to Hansen et al.; 5,962,112 to Haynes et al.; 6,093,665 to Sayovitz et al.; D375,844 to Edwards, et al.; D428,267 to Romano et al.; and D390,708 to Brown, which are incorporated herein in their entirety by reference thereto for all purposes.

The selection of an appropriate aperturing/embossing temperature (e.g., the temperature of a heated roll) will help melt and/soften the polymer composition of the film-nonwoven laminate at regions adjacent to the raised elements. The softened regions may then flow and become displaced during nipping, such as by pressure exerted by the raised elements. The displaced portions of the film/nonwoven laminate create the apertures.

To achieve aperture formation, the roll temperature and nip pressure may be selectively controlled. For example, one or more rolls may be heated to a surface temperature of from about 50° C. to about 160° C., in further embodiments from about 60° C. to about 140° C., and in even further embodiments, from about 70° C. to about 120° C. Likewise, the pressure exerted by rolls (“nip pressure”) upon the film-nonwoven laminate may range from about 75 to about 600 pounds per linear inch, in further embodiments from about 100 to about 400 pounds per linear inch, and in even further embodiments, from about 120 to about 200 pounds per linear inch. Of course, the residence time of the materials may influence the particular temperature and pressure employed.

As stated, another factor that influences aperture formation is the degree of tension in the film-nonwoven laminate during nipping. An increase in film-nonwoven laminate tension, for example, typically correlates to an increase in aperture size. Of course, a film-nonwoven laminate tension that is too high may adversely affect the integrity of the film-nonwoven laminate. Thus, in some embodiments of the present invention, a stretch ratio of about 1.5 or more, in further embodiments from about 2.5 to about 7.0, and in even further embodiments, from about 3.0 to about 5.5, may be employed to achieve the desired degree of tension in the film-nonwoven laminate. The stretch ratio in the machine direction may be determined by dividing the final length of the film-nonwoven laminate by its original length. The machine direction stretch ratio may also be approximately the same as the draw ratio, which may be determined by dividing the linear speed of the film-nonwoven laminate during nipping (e.g., speed of the nip rolls) by the linear speed at which the film-nonwoven laminate is formed (e.g., speed of casting rolls or blown nip rolls) or unwound. Alternatively, the film-nonwoven laminate may be tensioned/stretched in the cross direction, such as, for example by using a tentering frame to stretch the laminate in the cross direction. Such tenter frames are well known in the art and described, for instance, in U.S. Patent Application Publication No. 2004/0121687 to Morman, et al.

The film-nonwoven laminate may be “pre-stretched” (prior to nipping) by rolls rotating at different speeds of rotation so that the sheet is stretched to the desired stretch ratio in the machine direction. This uniaxially stretched film-nonwoven laminate may also be oriented in the cross-machine direction to form a “biaxially stretched” film-nonwoven laminate. The orientation temperature profile during the “pre-stretching” operation is generally below the melting point of one or more polymers in the film, but high enough to enable the film-nonwoven laminate to be drawn or stretched. For example, the film-nonwoven laminate may be stretched at a temperature from about 15° C. to about 50° C., in further embodiments from about 25° C. to about 40° C., and in even further embodiments, from about 30° C. to about 40° C. When “pre-stretched” in the manner described above, the degree of stretch during lamination may be increased, maintained, or slightly reduced (retracted) to desired degree of tension.

Upon nipping, the film-nonwoven laminate is apertured. The size and/or pattern of the resulting apertures generally correspond to the size and/or pattern of the raised elements. That is, the apertures and/or may have a length, width, aspect ratio, and orientation as described above. For example, the length dimension of the apertures may be from about 200 to about 5000 micrometers, in further embodiments from about 350 to about 4000 micrometers, and in even further embodiments, from about 500 to about 2500 micrometers. The width dimension of the apertures may likewise range from about 20 to about 500 micrometers, in further embodiments from about 40 to about 200 micrometers, and in even further embodiments, from about 50 to about 150 micrometers. In addition, the “aspect ratio” (the ratio of the length of an aperture to its width) may range from about 2 to about 100, in further embodiments from about 4 to about 50, and in even further embodiments, from about 5 to about 20. Similarly, the longitudinal axis of one or more of the apertures (longest dimension along a center line of the aperture) may be skewed relative to the machine direction of the film-nonwoven laminate, such as from about 30° to about 150°, in further embodiments from about 45° to about 135°, and in even further embodiments, from about 60° to about 120° relative to the machine direction of the film-nonwoven laminate. The apertures in the film are defined by an aperture perimeter formed by the raised elements (pins) of a patterned roll. Desirably, the aperture perimeter defines an aperture flap that extends at least partially into the aperture. In some embodiments the aperture flap may extend up to about 25% of the distance across the aperture, in further embodiments up to about 50% of the distance across the aperture, and in even further embodiments up to about 75% of the distance across the aperture.

Still referring to FIG. 1, the film-nonwoven laminate 80 is directed to a nip defined between rolls 90 for creating apertures in the film-nonwoven laminate. One or both of the rolls 90 may contain a plurality of raised elements and may be heated as described above. Upon nipping, apertures are created in the film-nonwoven laminate. The resulting apertured film-nonwoven laminate 32 may then be wound and stored on a take-up roll 95.

The resulting apertured film-nonwoven laminate 32 may then be wound and stored on a take-up roll 60. Optionally, the composite 32 is kept under tension, such as by using the same linear velocity for the roll 95 as the speed of one or more of the aperturing rolls 90. More preferably, however, the composite 32 is allowed to slightly retract prior to winding on to the take-up roll 95. This may be achieved by using a slower linear velocity for the roll 95. Because the film-nonwoven laminate 80 is tensioned prior to aperturing, it will retract toward its original machine direction length and become shorter in the machine direction. The resulting apertured film-nonwoven laminate 32 thus becomes extensible and elastic in the machine direction to the extent that the film-nonwoven laminate retracts after being stretched.

While not shown in FIG. 1, various additional potential processing and/or finishing steps known in the art, such as slitting, treating, printing graphics, etc., may be performed without departing from the spirit and scope of the invention. For instance, the apertured composite may optionally be mechanically stretched in the cross-machine and/or machine directions to enhance extensibility. In one embodiment, the apertured composite may be coursed through two or more rolls that have grooves in the CD and/or MD directions. Such grooved satellite/anvil roll arrangements are described in U.S. Patent Application Publication Nos. 2004/0110442 to Rhim, et al. and 2006/0151914 to Gerndt, et al., which are incorporated herein in their entirety by reference thereto for all purposes. For instance, the laminate may be coursed through two or more rolls that have grooves in the CD and/or MD directions. The grooved rolls may be constructed of steel or other hard material (such as a hard rubber).

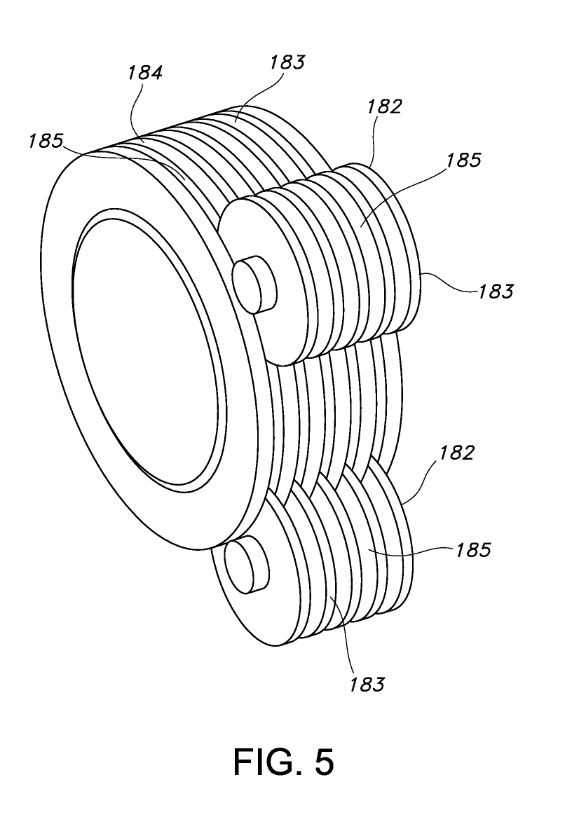

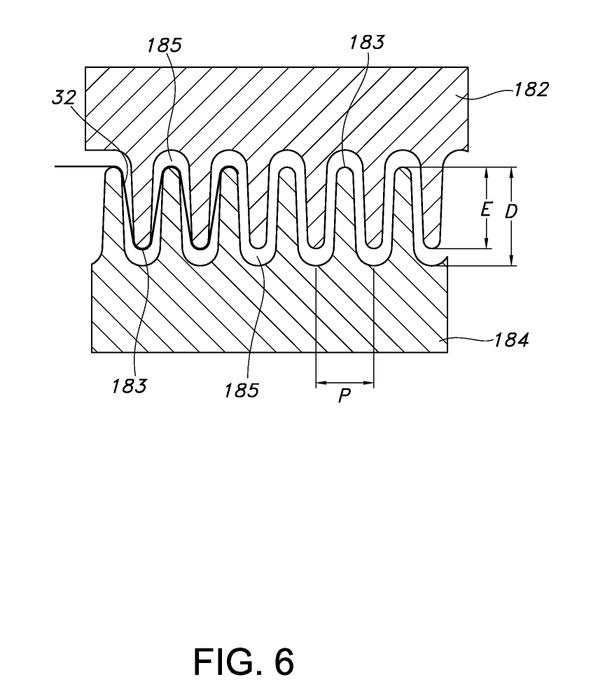

FIGS. 5-6 further illustrate the manner in which groove rolls may incrementally stretch the apertured composite. As shown, for example, satellite rolls 182 may engage an anvil roll 184, each of which include a plurality of ridges 183 defining a plurality of grooves 185 positioned across the grooved rolls in the cross-machine direction. The grooves 185 are generally oriented perpendicular to the direction of stretch of the material. In other words, the grooves 185 are oriented in the machine direction to stretch the apertured composite in the cross-machine direction. The grooves 185 may likewise be oriented in the cross-machine direction to stretch the apertured composite in the machine direction. The ridges 183 of satellite roll 182 intermesh with the grooves 185 of anvil roll 184, and the grooves 185 of satellite roll 182 intermesh with the ridges 183 of anvil roll 184.

The dimensions and parameters of the grooves 185 and ridges 183 may have a substantial effect on the degree of extensibility provided by the rolls 182 and 184. For example, the number of grooves 185 contained on a roll may generally range from about 3 and 15 grooves per inch, in further embodiments from about 5 and 12 laminate 32 thus becomes extensible and elastic in the machine direction to the extent that the film-nonwoven laminate retracts after being stretched.

While not shown in FIG. 1, various additional potential processing and/or finishing steps known in the art, such as slitting, treating, printing graphics, etc., may be performed without departing from the spirit and scope of the invention. For instance, the apertured composite may optionally be mechanically stretched in the cross-machine and/or machine directions to enhance extensibility. In one laminate 32 thus becomes extensible and elastic in the machine direction to the extent that the film-nonwoven laminate retracts after being stretched.

While not shown in FIG. 1, various additional potential processing and/or finishing steps known in the art, such as slitting, treating, printing graphics, etc., may be performed without departing from the spirit and scope of the invention. For instance, the apertured composite may optionally be mechanically stretched in the cross-machine and/or machine directions to enhance extensibility. In one about 0.8 centimeters, and in further embodiments, from about 0.15 to about 0.4 centimeters. Regardless, the apertured composite 32 (FIG. 1) may be stretched in one or more directions at a stretch ratio of from about 1.5 to about 8.0, in further embodiments by at least about 2.0 to about 6.0, and in even further embodiments, from about 2.5 to about 4.5. If desired, heat may be applied to the composite just prior to or during the application of incremental stretch to cause it to relax somewhat and ease extension. Heat may be applied by any suitable method known in the art, such as heated air, infrared heaters, heated nipped rolls, or partial wrapping of the laminate around one or more heated rolls or steam canisters, etc. Heat may also be applied to the grooved rolls themselves. It should also be understood that other grooved roll arrangement are equally suitable, such as two grooved rolls positioned immediately adjacent to one another.

Besides the above-described grooved rolls, other techniques may also be used to mechanically stretch the apertured composite in one or more directions. For example, the apertured composite may be passed through a tenter frame that stretches the composite. The composite may also be necked. Suitable techniques necking techniques are described in U.S. Pat. Nos. 5,336,545, 5,226,992, 4,981,747 and 4,965,122 to Morman, as well as U.S. Patent Application Publication No. 2004/0121687 to Morman, et al., all of which are incorporated herein in their entirety by reference thereto for all purposes.

The apertured film/nonwoven composite of the present invention may be used in a wide variety of applications. As noted above, for example, the apertured film/nonwoven nonwoven composite may be used in an absorbent article. An “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins), swim wear, baby wipes, and so forth; medical absorbent articles, such as garments, fenestration materials, underpads, bedpads, bandages, absorbent drapes, and medical wipes; food service wipers; clothing articles; and so forth. Materials and processes suitable for forming such absorbent articles are well known to those skilled in the art. Absorbent articles may include a substantially liquid-impermeable layer (e.g., outer cover), a liquid-permeable layer (e.g., bodyside liner, surge layer, etc.), and an absorbent core.

In one particular embodiment, the apertured film/nonwoven composite of the present invention may be used to form a liquid-permeable layer (e.g., bodyside liner, surge layer) of the absorbent article. As described above, apertures are formed through both the elastic film and nonwoven web material. The existence of the apertures through the fibers of the nonwoven web enhances the ability of the composite to be employed as a liquid-permeable layer in an absorbent article. Namely, because the nonwoven web material is apertured adjacent to the film apertures, a liquid may more readily flow through the nonwoven web material and into the film aperture.

Besides liquid-permeable materials (e.g., liners, surge layers, etc.), the apertured film/nonwoven composite of the present invention may have a wide variety of other uses, such as in providing an elastic waist, leg cuff/gasketing, stretchable ear, side panel, outer cover, or any other component in which elastic properties are desirable.

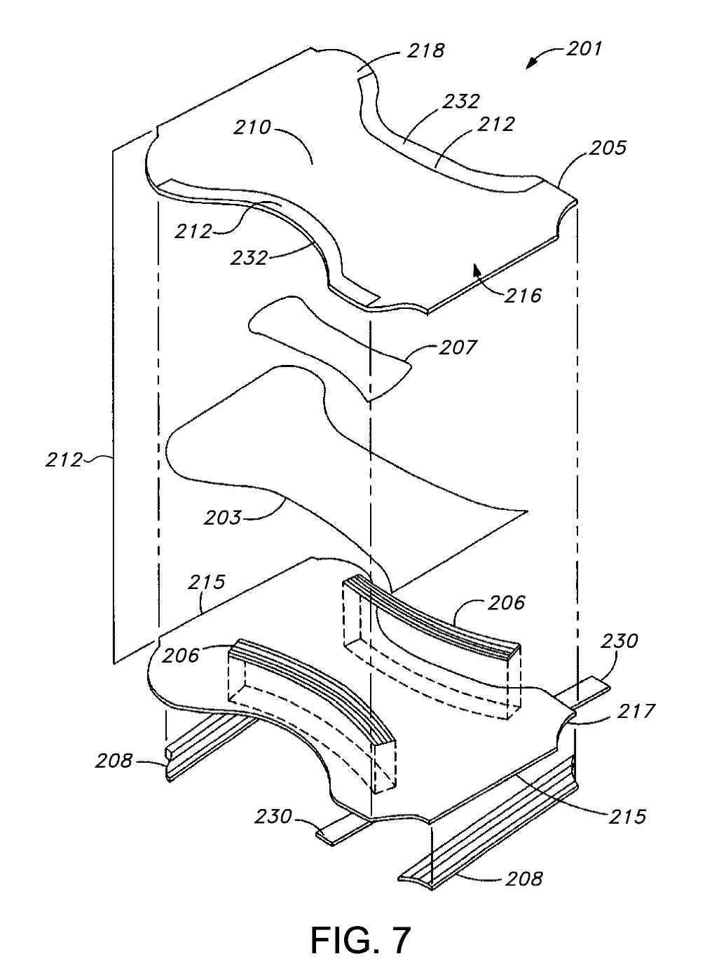

Various embodiments of an absorbent article that may be formed according to the present invention will now be described in more detail. For purposes of illustration only, an absorbent article is shown in FIG. 7 as a diaper 201. However, as noted above, the invention may be embodied in other types of absorbent articles, such as incontinence articles, sanitary napkins, diaper pants, feminine napkins, children's training pants, and so forth. In the illustrated embodiment, the diaper 201 is shown as having an hourglass shape in an unfastened configuration. However, other shapes may of course be utilized, such as a generally rectangular shape, T-shape, or I-shape. As shown, the diaper 201 includes a chassis 202 formed by various components, including an outer cover 217, bodyside liner 205, absorbent core 203, and surge layer 207. It should be understood, however, that other layers may also be used in the present invention. Likewise, one or more of the layers referred to in FIG. 7 may also be eliminated in certain embodiments of the present invention.

The bodyside liner 205 is generally employed to help isolate the wearer's skin from liquids held in the absorbent core 203. For example, the liner 205 presents a bodyfacing surface that is typically compliant, soft feeling, and non-irritating to the wearer's skin. Typically, the liner 205 is also less hydrophilic than the absorbent core 203 so that its surface remains relatively dry to the wearer. As indicated above, the liner 205 may be liquid-permeable to permit liquid to readily penetrate through its thickness. Exemplary liner constructions that contain a nonwoven web are described in U.S. Pat. Nos. 5,192,606 to Proxmire, et al.; 5,702,377 to Collier, IV, et al.; 5,931,823 to Stokes, et al.; 6,060,638 to Paul, et al.; and 6,150,002 to Varona, as well as U.S. Patent Application Publication Nos. 2004/0102750 to Jameson; 2005/0054255 to Morman, et al.; and 2005/0059941 to Baldwin, et al., all of which are incorporated herein in their entirety by reference thereto for all purposes. In one particular embodiment, the liner includes the apertured film/nonwoven composite of the present invention.

As illustrated in FIG. 7, the diaper 201 may also include a surge layer 207 that helps to decelerate and diffuse surges or gushes of liquid that may be rapidly introduced into the absorbent core 203. Desirably, the surge layer 207 rapidly accepts and temporarily holds the liquid prior to releasing it into the storage or retention portions of the absorbent core 203. In the illustrated embodiment, for example, the surge layer 207 is interposed between an inwardly facing surface 216 of the bodyside liner 205 and the absorbent core 203. Alternatively, the surge layer 207 may be located on an outwardly facing surface 218 of the bodyside liner 205. The surge layer 207 is typically constructed from highly liquid-permeable materials. Examples of suitable surge layers are described in U.S. Pat. No. 5,486,166 to Ellis, et al. and U.S. Pat. No. 5,490,846 to Ellis, et al., which are incorporated herein in their entirety by reference thereto for all purposes. In one particular embodiment, the surge layer 207 includes the apertured film/nonwoven composite of the present invention.

The outer cover 217 is typically formed from a material that is substantially impermeable to liquids. For example, the outer cover 217 may be formed from a thin plastic film or other flexible liquid-impermeable material. In one embodiment, the outer cover 217 is formed from a polyethylene film having a thickness of from about 0.01 millimeter to about 0.05 millimeter. The film may be impermeable to liquids, but permeable to gases and water vapor (i.e., “breathable”). This permits vapors to escape from the absorbent core 203, but still prevents liquid exudates from passing through the outer cover 217. If a more cloth-like feeling is desired, the outer cover 217 may be formed from a polyolefin film laminated to a nonwoven web. For example, a stretch-thinned polypropylene film may be thermally laminated to a spunbond web of polypropylene fibers.

Besides the above-mentioned components, the diaper 201 may also contain various other components as is known in the art. For example, the diaper 201 may also contain a substantially hydrophilic tissue wrapsheet (not illustrated) that helps maintain the integrity of the fibrous structure of the absorbent core 203. The tissue wrapsheet is typically placed about the absorbent core 203 over at least the two major facing surfaces thereof, and composed of an absorbent cellulosic material, such as creped wadding or a high wet-strength tissue. The tissue wrapsheet may be configured to provide a wicking layer that helps to rapidly distribute liquid over the mass of absorbent fibers of the absorbent core 203. The wrapsheet material on one side of the absorbent fibrous mass may be bonded to the wrapsheet located on the opposite side of the fibrous mass to effectively entrap the absorbent core 203. Furthermore, the diaper 201 may also include a ventilation layer (not shown) that is positioned between the absorbent core 203 and the outer cover 217. When utilized, the ventilation layer may help insulate the outer cover 217 from the absorbent core 203, thereby reducing dampness in the outer cover 217. Examples of such ventilation layers may include a nonwoven web laminated to a breathable film, such as described in U.S. Pat. No. 6,663,611 to Blaney, et al., which is incorporated herein in its entirety by reference thereto for all purposes.

In some embodiments, the diaper 201 may also include a pair of side panels (or ears) (not shown) that extend from the side edges 232 of the diaper 201 into one of the waist regions. The side panels may be integrally formed with a selected diaper component. For example, the side panels may be integrally formed with the outer cover 217 or from the material employed to provide the top surface. In alternative configurations, the side panels may be provided by members connected and assembled to the outer cover 217, the top surface, between the outer cover 217 and top surface, or in various other configurations. If desired, the side panels may be elasticized or otherwise rendered elastomeric by use of the elastic film/nonwoven composite of the present invention. Examples of absorbent articles that include elasticized side panels and selectively configured fastener tabs are described in PCT Patent Application WO 95/16425 to Roessler; U.S. Pat. No. 5,399,219 to Roessler et al.; U.S. Pat. No. 5,540,796 to Fries; and U.S. Pat. No. 5,595,618 to Fries, each of which is incorporated herein in its entirety by reference thereto for all purposes.

As representatively illustrated in FIG. 7, the diaper 201 may also include a pair of containment flaps 212 that are configured to provide a barrier and to contain the lateral flow of body exudates. The containment flaps 212 may be located along the laterally opposed side edges 232 of the bodyside liner 205 adjacent the side edges of the absorbent core 203. The containment flaps 212 may extend longitudinally along the entire length of the absorbent core 203, or may only extend partially along the length of the absorbent core 203. When the containment flaps 212 are shorter in length than the absorbent core 203, they may be selectively positioned anywhere along the side edges 232 of diaper 201 in a crotch region 210. In one embodiment, the containment flaps 212 extend along the entire length of the absorbent core 203 to better contain the body exudates. Such containment flaps 212 are generally well known to those skilled in the art. For example, suitable constructions and arrangements for the containment flaps 212 are described in U.S. Pat. No. 4,704,116 to Enloe, which is incorporated herein in its entirety by reference thereto for all purposes.

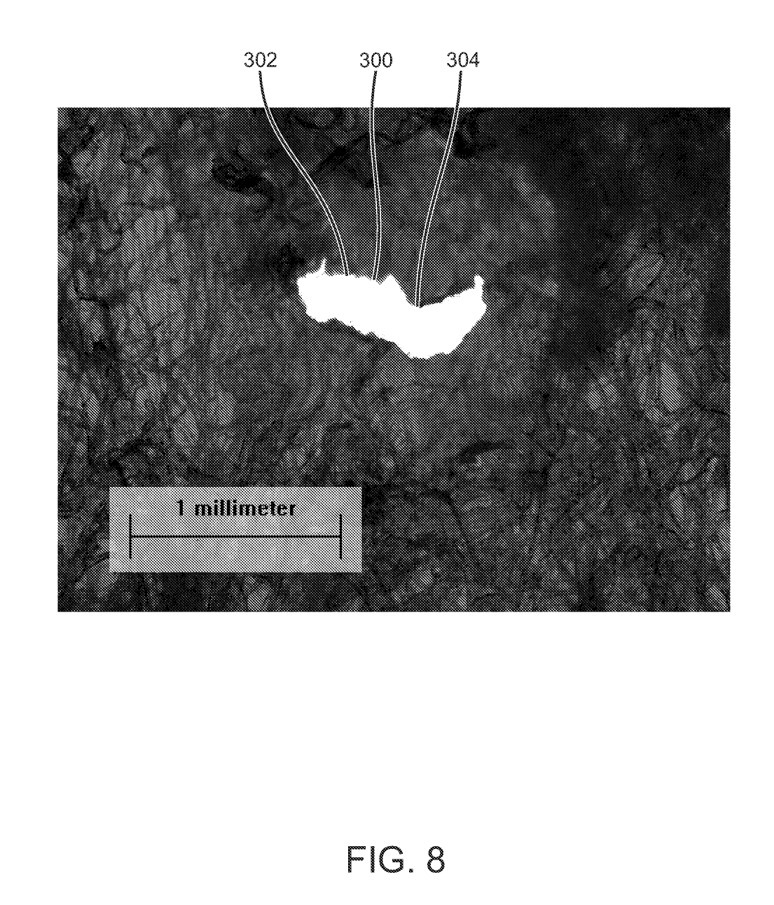

To provide improved fit and to help reduce leakage of body exudates, the diaper 201 may be elasticized with suitable elastic members, as further explained below. For example, as representatively illustrated in FIG. 8, the diaper 201 may include leg elastics 206 constructed to operably tension the side margins of the diaper 201 to provide elasticized leg bands which can closely fit around the legs of the wearer to reduce leakage and provide improved comfort and appearance. Waist elastics 208 may also be employed to elasticize the end margins of the diaper 201 to provide elasticized waistbands. The waist elastics 208 are configured to provide a resilient, comfortably close fit around the waist of the wearer. The elastic film/nonwoven composite of the present invention is suitable for use as the leg elastics 206 and waist elastics 208. Exemplary of such materials are laminate sheets that either comprise or are adhered to the outer cover 217 so that elastic constrictive forces are imparted thereto.

The diaper 201 may also include one or more fasteners 230. For example, two flexible fasteners 230 are illustrated in FIG. 7 on opposite side edges of waist regions to create a waist opening and a pair of leg openings about the wearer. The shape of the fasteners 230 may generally vary, but may include, for instance, generally rectangular shapes, square shapes, circular shapes, triangular shapes, oval shapes, linear shapes, and so forth. The fasteners may include, for instance, a hook-and-loop material, buttons, pins, snaps, adhesive tape fasteners, cohesives, fabric-and-loop fasteners, etc. In one particular embodiment, each fastener 230 includes a separate piece of hook material affixed to the inside surface of a flexible backing.

The various regions and/or components of the diaper 201 may be assembled together using any known attachment mechanism, such as adhesive, ultrasonic, thermal bonds, etc. Suitable adhesives may include, for instance, hot melt adhesives, pressure-sensitive adhesives, and so forth. When utilized, the adhesive may be applied as a uniform layer, a patterned layer, a sprayed pattern, or any of separate lines, swirls or dots. In the illustrated embodiment, for example, the outer cover 217 and bodyside liner 205 are assembled to each other and to the absorbent core 203 using an adhesive. Alternatively, the absorbent core 203 may be connected to the outer cover 217 using conventional fasteners, such as buttons, hook and loop type fasteners, adhesive tape fasteners, and so forth. Similarly, other diaper components, such as the leg elastic members 206, waist elastic members 208 and fasteners 230, may also be assembled into the diaper 201 using any attachment mechanism.

Although various configurations of a diaper have been described above, it should be understood that other diaper and absorbent article configurations are also included within the scope of the present invention. In addition, the present invention is by no means limited to diapers. In fact, any other absorbent article may be formed in accordance with the present invention, including, but not limited to, other personal care absorbent articles, such as training pants, absorbent underpants, adult incontinence products, feminine hygiene products (e.g., sanitary napkins), swim wear, baby wipes, and so forth; medical absorbent articles, such as garments, fenestration materials, underpads, bandages, absorbent drapes, and medical wipes; food service wipers; clothing articles; and so forth. Several examples of such absorbent articles are described in U.S. Pat. Nos. 5,649,916 to DiPalma, et al.; 6,110,158 to Kielpikowski; 6,663,611 to Blaney, et al., which are incorporated herein in their entirety by reference thereto for all purposes. Still other suitable articles are described in U.S. Patent Application Publication No. 2004/0060112 A1 to Fell et al., as well as U.S. Pat. Nos. 4,886,512 to Damico et al.; 5,558,659 to Sherrod et al.; 6,888,044 to Fell et al.; and 6,511,465 to Freiburger et al., all of which are incorporated herein in their entirety by reference thereto for all purposes.

The present invention may be better understood with reference to the following examples.

### EXAMPLES

The ability to form an apertured elastic nonwoven composite was demonstrated at a number of different processing conditions. The film used in the composite contained approximately 90-95 wt % of an elastomer composition and 5-10 wt % of a thermoplastic composition as a skin layer. The elastomer composition contained 48 wt % KRATON® MD6716 (Kraton Polymers, LLC of Houston Tex.), 48 wt % KRATON® MD6673 (Kraton Polymers, LLC), and 4 wt % SCC 4837 (“Standridge Color Corporation, Social Circle, Ga.). KRATON® MD6716 contains approximately 75 wt % of a styrene-ethylene-butylene-styrene (“SEBS”) block copolymer, tackifier, and polyethylene wax, and has a target melt flow rate of 7 g/10 min (200° C., 5 kg). KRATON® MD6673 contains 68 wt % of a styrene-ethylene-butylene-styrene block copolymer (KRATON® MD6937), 20 wt % REGALREZ™ 1126 (Eastman Chemical) and 12 wt % EPOLENE™ C-10 polyethylene wax (Eastman Chemical). SCC 4837 is a pigment containing titanium dioxide blended with polyethylene. The thermoplastic composition contained 48 wt % PP3155 (ExxonMobil Chemical Company, 48 wt % DOWLEX™ 2517 (The Dow Chemical Company), and 4 wt % SCC 4837. PP3155 is a polypropylene homopolymer resin having a melt flow rate of 36 g/10 min (230° C., 2.16 kg) and a density of 0.9 g/cm³. DOWLEX™ 2517 is a linear low density polyethylene resin with a melt index of 25 g/10 min (190° C., 2.16 kg), a density of 0.917 g/cm³, and a melting point of 255° F. The composite also included two facings between which the film was sandwiched. Both facings of the composite were prepared with a 17 gsm meltblown web containing 60 wt % DNDA 1082 NT-7 (The Dow Chemical Company) and 40 wt % VISTAMAXX™ 2330 (ExxonMobil Chemical Company). DNDA 1082 NT-7 is a linear low density polyethylene resin with a melt index of 155 g/10 min (190° C., 2.16 kg), a density of 0.933 g/cm³, and a melting point of 257° F. VISTAMAXX™ 2330 is a polyolefin copolymer/elastomer with a melt flow rate of 285 g/10 min and a density of 0.868 g/cm³.

The polymers for the film and meltblown layers were compounded by weighing appropriate portions of pellets of each polymer, combining them into one container, and mixing them together by stirring. After compounding, the elastomeric film was formed to a basis weight of 45 gsm using a 20″ wide Randcastle co-extruding film die. In this case, the elastomeric component of the film was extruded in the center of the film, and the thermoplastic component was extruded on either side of the elastomeric layer to form a sandwich structure (i.e. an A-B-A film with the thermoplastic as layer A and the elastomer as layer B). Both meltblown facings were prepared to a basis weight of 17 gsm using a 20″ wide meltblown system having 30 capillaries per inch of die width.

To form the composite, the bottom meltblown facing was unwound onto extrusion coating roll. The film was then extruded onto the meltblown web. The other meltblown web was then nipped onto the film/meltblown layer while the film was still molten to form a MB/film/MB composite. The composite was then directed into a nip having a patterned roll and a smooth anvil roll. Two different pin patterns were used to aperture the MB/film/MB laminate materials. Pattern #1 (Codes 1-6) had 32 round pins per square inch that covered 9.8% of the roll surface area. Pattern #2 (Codes 7-15) had 53 diamond-shaped raised elements per square inch, the elements covering 8.1% of the roll surface area. The material was then wound. The nipping/aperturing conditions are listed in Table 1 for a number of different process conditions.

FIGS. 8-11 show exemplary scanning electron microphotographs of the resulting samples for Code 1. FIGS. 8-10, for instance, show the approximately crescent-shaped apertures 300 defined by an aperture perimeter 302 formed by the pins of a patterned roll. FIGS. 8-11, for instance, show a flap 304 defined by the aperture perimeter 302 and extending at least partially into the aperture 300.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto. As used herein, the term “comprising” is inclusive or open-ended and does not exclude additional unrecited elements, compositional components, or method steps. In addition, it should be noted that any given range presented herein is intended to include any and all lesser included ranges. For example, a range of from 45-90 would also include 50-90; 45-80; 46-89 and the like.

TABLE 1

| | Roll | Roll | Roll | Nip | Stretch | |
|---|---|---|---|---|---|---|
| Code # | Temp | Pressure | Speed | Speed | Ratio | Observations |
| 1 | 210° F. | 25 psi | 10 fpm | 10fpm | 1 | Apertures observed. |
| 2 | 210° F. | 25 psi | 30 fpm | | | Matl failed |
| 3 | 200° F. | 25 psi | 30 fpm | 10 fpm | 3 | Apertures observed in matl |
| 4 | 200° F. | 40 psi | 30 fpm | 10 fpm | 3 | Apertures observed in matl |
| 5 | 200° F. | 40 psi | 30 fpm | 10 fpm | 3 | Apertures observed in matl |
| 6 | 200° F. | 40 psi | 35 fpm | 10 fpm | 3.5 | Apertures observed in this |
| | | | | | | matl |
| 7 | 200° F. | 40 psi | 16 fpm | 10 fpm | 1.6 | Small number of apertures |
| | | | | | | observed randomly |
| | | | | | | throughout material. |
| 8 | 200° F. | 30 psi | 16 fpm | 10 fpm | 1.6 | Small number of apertures |
| | | | | | | observed randomly |
| | | | | | | throughout material. |
| 9 | 200° F. | 20 psi | 13 fpm | 10 fpm | 1.3 | Small number of apertures |
| | | | | | | observed randomly |
| | | | | | | throughout material. |
| 10 | 180° F. | 20 psi | 10 fpm | 10 fpm | 1 | No apertures observed. |
| 11 | 212° F. | 20 psi | 10 fpm | 10 fpm | 1 | Small number of apertures |
| | | | | | | observed randomly |
| | | | | | | throughout material. |
| 12 | 212° F. | 30 psi | 10 fpm | 10 fpm | 1 | Small number of apertures |
| | | | | | | observed randomly |
| | | | | | | throughout material. |
| 13 | 212° F. | 40 psi | 10 fpm | 10 fpm | 1 | Small number of apertures |
| | | | | | | observed randomly |
| | | | | | | throughout material. |
| 14 | 212° F. | 45 psi | 10 fpm | 10 fpm | 1 | Small number of apertures |
| | | | | | | observed randomly |
| | | | | | | throughout material. |
| 15 | 217° F. | 45 psi | 10 fpm | 10 fpm | 1 | Not many apertures observed |
| | | | | | | in this material. |

What is claimed is:

1. A method of forming a nonwoven composite, the method comprising:
forming an extensible fibrous nonwoven web in a machine direction;
forming an elastic film in the machine direction on a surface of the extensible fibrous nonwoven web to form a film/nonwoven laminate;
stretching the film/nonwoven laminate;
passing the film and a nonwoven web material through a nip formed by at least one patterned roll; and
at the nip, concurrently forming apertures in the elastic film and the extensible fibrous nonwoven web to form an apertured film/nonwoven composite.

2. The method of claim 1, wherein the film is under tension at a stretch ratio of about 1.5 or more in the machine direction at the nip.

3. The method of claim 1, wherein the film comprises an elastomeric semi-crystalline polyolefin.

4. The method of claim 3, wherein the semi-crystalline polyolefin is an ethylene/α-olefin copolymer, propylene/α-olefin copolymer, or a combination thereof.

5. The method of claim 3, wherein the semi-crystalline polyolefin is single-site catalyzed.

6. The method of claim 1, wherein the film comprises an elastomeric block copolymer.

7. The method of claim 1, wherein the stretch ratio is from about 1.5 to about 5.0.

8. The method of claim 1, wherein the stretch ratio is from about 2.0 to about 4.0.

9. The method of claim 1, wherein the film/nonwoven laminate is stretched prior to passing through the nip.

10. The method of claim 1, wherein at least one of the apertures has a length of from about 350 to about 4000 micrometers.

11. The method of claim 1, wherein the roll is patterned with raised elements.

12. The method of claim 11, wherein at least one of the raised elements is oriented from about 30° to about 150° relative to the machine direction.

13. The method of claim 11, wherein at least one of the raised elements is oriented from about 45° to about 135° relative to the machine direction.

14. The method of claim 1, wherein the nip is formed between two rolls.

15. The method of claim 14, wherein at least one of the rolls is heated to a surface temperature of from about 50° C. to about 160° C.

16. The method of claim 14, wherein at least one of the rolls is heated to a surface temperature of from about 70° C. to about 120° C.

17. The method of claim 14, wherein a pressure from about 75 to about 600 pounds per linear inch is applied at the nip.

18. The method of claim 14, wherein a pressure from about 120 to about 200 pounds per linear inch is applied at the nip.

19. The method of claim 1, wherein the extensible fibrous nonwoven web material contains spunbond fibers, meltblown fibers, staple fibers, or a combination thereof.

20. The method of claim 1, wherein the extensible fibrous nonwoven web material comprises a polyolefin.

21. The method of claim 20, wherein the polyolefin is polypropylene.

22. The method of claim 1, further comprising allowing the composite to retract in the machine direction prior to or during winding onto a roll.

23. The method of claim 1, further comprising mechanically stretching the apertured composite in at least the cross-machine direction.

24. A film/nonwoven laminate formed by the method of claim 1.

25. An absorbent article comprising an outer cover, a bodyside liner joined to the outer cover, and an absorbent core positioned between the outer cover and the bodyside liner, wherein the absorbent article comprises the film/nonwoven laminate of claim 24.

26. An absorbent article comprising an outer cover, a liner and an absorbent core forming a chassis, at least a portion of the chassis comprising the nonwoven composite of claim 24.

27. The absorbent article of claim 25, wherein the bodyside liner includes the nonwoven composite.

28. The absorbent article of claim 25, the absorbent article further comprising a surge layer comprising the nonwoven composite.

29. The absorbent article of claim 25, further comprising a waist band, a leg band, or both, that comprises the nonwoven composite.

30. The method of claim 1, wherein the stretching occurs in a direction selected from the group consisting of a machine direction and a cross direction.
